(19) United States
(12) Patent Application Publication
(10) Pub. No.: US 2013/0323231 A1
(43) Pub. Date: Dec. 5, 2013

(54) TREATMENT AND PROGNOSIS OF SOLID TUMOUR CANCERS

(71) Applicants: Caoimhin Concannon, Dublin (IE); Jochen Prehn, Dublin (IE)

(72) Inventors: Caoimhin Concannon, Dublin (IE); Jochen Prehn, Dublin (IE)

(73) Assignee: ROYAL COLLEGE OF SURGEONS IN IRELAND, Dublin (IE)

(21) Appl. No.: 13/985,660

(22) PCT Filed: Mar. 30, 2012
(86) PCT No.: PCT/EP2012/055815

§ 371 Date: Aug. 15, 2013

Related U.S. Application Data
(60) Provisional application No. 61/469,978, filed on Mar. 31, 2011.

(51) Int. Cl. A61K 31/7088; A61K 45/06

(52) U.S. Cl. CPC A61K 31/7088; A61K 45/06
USPC 424/130.1; 424/649; 436/501; 514/44 A

(57) ABSTRACT

The invention is based on the finding that the human RPT4 protein/gene can function as a therapeutic target for solid tumour type cancers, especially solid tumours of the colon, and that reducing the abundance of RPT4 in tumour cells causes a significant increase in cancer cell death, and potently decreases the survival and proliferation of cancer cells in tumour growth assays (FIGS. 3 and 4). A further, but linked, aspect of the invention is based on the finding that inhibitors of RPT4 significantly decrease the viability of chemotherapeutic-resistant tumours, especially solid tumours, especially colorectal solid tumours (FIG. 5). A further, but linked, aspect is based on the finding that the efficacy of conventional chemotherapeutic therapy, for example 5-FU/oxaliplatin therapy, is significantly improved when combined with treatment with a RPT4 inhibitor (FIG. 6). A further, but linked, aspect of the invention is based on the finding that levels of RPT4 in solid tumours such as colorectal cancer can function as a prognostic variable of outcome/survival (FIG. 2).

### INTRODUCTION

The invention relates to a method for the treatment of a solid tumour, especially solid tumours of the colon, in an individual in need thereof. The invention also relates to a method of prognosis of outcome in an individual having established solid tumour cancer, especially solid tumour of the colon.

Colorectal cancer (CRC) is one of the most common forms of cancer in both males and females and a leading cause of cancer related deaths. The current treatment methods for advanced CRC are the use of aggressive and invasive treatments. Surgical resection is the mainstay of treatment of CRC. For small tumours localised to the bowel wall (Stage I), resection offers an excellent chance of complete cure, with an 80-95% 5-year survival. However when tumours invade the bowel wall (Stage II) and there is an involvement of lymph node metastasis (Stage III) the survival rate is radically reduced (30-60% 5-year survival). Unfortunately the vast majority of patients present in these more advanced stages. Adjuvant chemotherapy (plus radiotherapy) plays a definite role in colorectal carcinoma and has been shown to improve disease free and overall survival of patients with resected Stage III CRC. 5-FU/Oxaliplatin- or 5-FU/Irinotecan based chemotherapy regimens are the standard treatment for CRC in both the adjuvant and advanced disease settings. However, drug resistance to these genotoxic drugs is thought to cause treatment failure in up to 90% of patients with metastatic cancer. In addition to the problem of drug resistance, standard genotoxic chemotherapeutics such as 5-FU, Irinotecan, and Oxaliplatin act relatively unspecifically and target all cells in the human body. This well-known problem causes dangerous side-effects that greatly impact on the patients and limit the use of high-dose chemotherapy.

Proteins are molecules in the body that are required for numerous functions that help to maintain a healthy body. Cellular homeostasis is tightly governed by a balance between protein synthesis and degradation. Indeed many diseases result from an interruption of this equilibrium. The proteasome, a multi-catalytic enzyme complex, has a crucial role in the degradation of intracellular proteins, many of which are critically involved in the cell cycle modulation, DNA repair, apoptosis, and neoplasia (Eldridge and O'Brien (2010) Cell Death Diff. 17: 4-13). The functional active complex is termed the 26 S proteasome and is found in both the nucleus and cytoplasm of eukaryotic cells. This complex is composed of a 20S catalytic core which is the site of protein destruction and degradation and two 19S regulatory caps. Current alternative approaches for the treatment of cancer include the proteasome inhibitor, bortezomib, which has been approved for the treatment of multiple myeloma, and is in clinical development for the treatment of solid tumours. However, bortezomib inhibits in a non-selective manner all chymotrypsin-like activity of the 20S core, and is not selective for cancer cells (see Bedford et al; (2011) Nature Reviews Drug Discovery 10: 29-46). The non-selective inhibition of all chymotrypsin-like activities of Bortezomib is also responsible for the dosage-limiting side effects of Bortezomib, such as the development of polyneuropathies (Ravaglia et al. (2008) Clin. Neurophysiol. 119: 2507-12).

It is an object of the invention to overcome at least one of the above-referenced problems.

### STATEMENTS OF INVENTION

Broadly, the present invention is based on the targeting of a sub-unit of the 19 S regulatory complex, know as Rpt 4 (hereafter referred to as “RPT4”), which has been identified as being expressed at higher levels than other proteasomal subunits, in tumour tissue of colorectal cancer patients when compared to normal tissue of the same patients. The invention is based on the finding that this protein/gene can function as a therapeutic target for solid tumour type cancers, especially solid tumours of the colon, and that reducing the abundance of RPT4 in tumour cells causes a significant increase in cancer cell death, and potently decreases the survival and proliferation of cancer cells in tumour growth assays (FIGS. 3 and 4). A further, but linked, aspect of the invention is based on the finding that inhibitors of RPT4 significantly decrease the viability of chemotherapeutic-resistant tumours, especially solid tumours, especially colorectal solid tumours (FIG. 5). A further, but linked, aspect is based on the finding that the efficacy of conventional chemotherapeutic therapy, for example 5-FU/oxaliplatin therapy, is significantly improved when combined with treatment with a RPT4 inhibitor (FIG. 6). A further, but linked, aspect of the invention is based on the finding that levels of RPT4 in solid tumours such as colorectal cancer can function as a prognostic variable of outcome/survival (FIG. 2).

In a first aspect, the invention relates to a method for the prevention or treatment of a chemotherapeutic-resistant cancer, especially a chemotherapeutic-resistant solid tumor cancer, in an individual in need thereof, the method comprising a step of administering to the individual a therapeutically effective amount of a RPT4 inhibitor. Also provided is a pharmaceutical composition comprising a low molecular weight RPT4 inhibitor, and a pharmaceutically acceptable excipient.

In a second aspect, the invention relates to a method for the prevention or treatment of a cancer, especially a solid tumour cancer, in an individual in need thereof, the method comprising a step of administering to the individual a therapeutically effective amount of a RPT4 inhibitor and a therapeutically effective amount of a conventional chemotherapeutic agent. Also provided is a pharmaceutical composition comprising a low molecular weight RPT4 inhibitor, a conventional solid tumour chemotherapeutic agent, and a pharmaceutically acceptable excipient.

In a third aspect, the invention relates to a method for increasing the sensitivity of a tumour, especially a solid tumour, for example a solid tumour of the colon, breast, ovary, or prostate gland, to a conventional chemotherapeutic agent in an individual in need thereof, the method comprising a step of administering to the individual a therapeutically effective amount of a RPT4 inhibitor.

In a fourth aspect, the invention relates to a method of predicting outcome in a patient with established solid tumour cancer comprising a step of assessing the abundance of RPT4 expression in tumour tissue and correlating the abundance of RPT4 expression with outcome. In one embodiment, increased RPT4 expression (typically in node positive patients) in tumour tissue correlates with low chance of disease free survival and death. Typically, the patient has a Stage III cancer. Typically the cancer is CRC.

In a fifth aspect, the invention provides a method of identifying a cancer therapeutic, typically a therapeutic capable of treating a solid tumour such as a CRC, breast, ovarian or prostate tumour, comprising a step of assaying a candidate agent for RPT4 inhibitory activity. In order to screen for RPT4 inhibitory activity, it is possible to utilize a RPT4 ATPase assay, for example one based on high-throughput screening of luciferase activity. This assay will typically rely on the principal of monitoring luciferase expression as an indirect measurement of ATP levels remaining following incubation with recombinant RPT4 protein and subsequent screening of compound library for hits that may affect the ATP levels.

In a sixth aspect, the invention relates to the use of a RPT4 inhibitor as a medicament, especially use of a low molecular weight inhibitor of RPT4 expression or a RPT4-specific antibody (or antibody fragment) as a medicament.

In a seventh aspect, the invention relates to a pharmaceutical composition comprising a RPT4 inhibitor

In an eighth aspect, the invention relates to a pharmaceutical composition (provided in the form of a unit dose or a kit of parts) comprising a RPT4 inhibitor (especially a low molecular weight inhibitor of RPT4 expression), a conventional chemotherapeutic agent, and a pharmaceutically acceptable carrier.

In a ninth aspect, the invention relates to a method for the prevention or treatment of a cancer, especially a solid tumor cancer, in an individual in need thereof, the method comprising a step of administering to the individual a therapeutically effective amount of a RPT4 inhibitor.

### BRIEF DESCRIPTION OF THE FIGURES

FIG. 1: (A) Western Blot of expression levels for RPT4 in colon tumour samples (T) and normal adjacent mucosal (N) specimens which shows a significant increase in protein expression levels of the proteasomal subunit Rpt4 in colonic tumour tissue compared to normal tissue. (B) Western Blot of expression levels of ubiquitin proteins in colon tumour samples (T) and normal adjacent mucosal (N) specimens which demonstrates no significant modulation of expression levels of ubiquitinylated proteins in colonic tumour tissue compared to normal tissue.

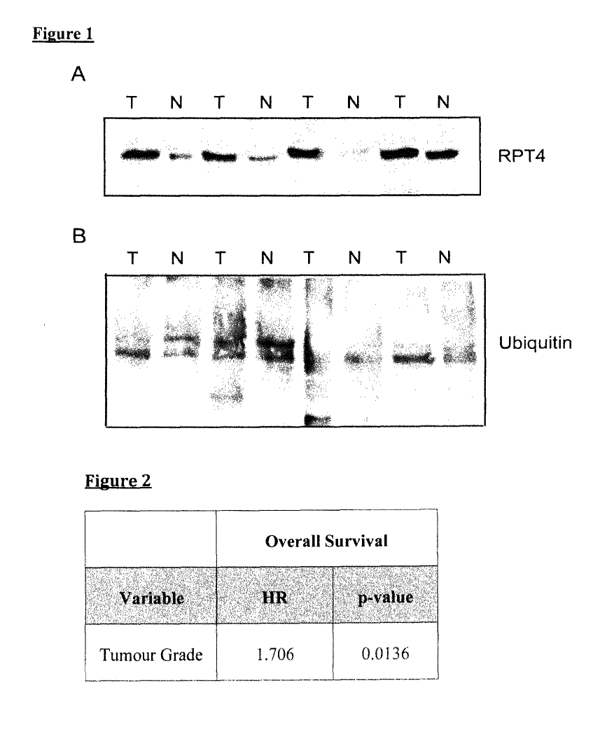

FIG. 2: The intensity of Rpt4 staining can serve as a prognostic marker in colon cancer patients. Cox proportional hazard analysis demonstrated that in Stage III patients with more advanced tumour grade, increased Rpt4 staining significantly predicted those who were more likely to die from the disease (HR: Hazard Ratio).

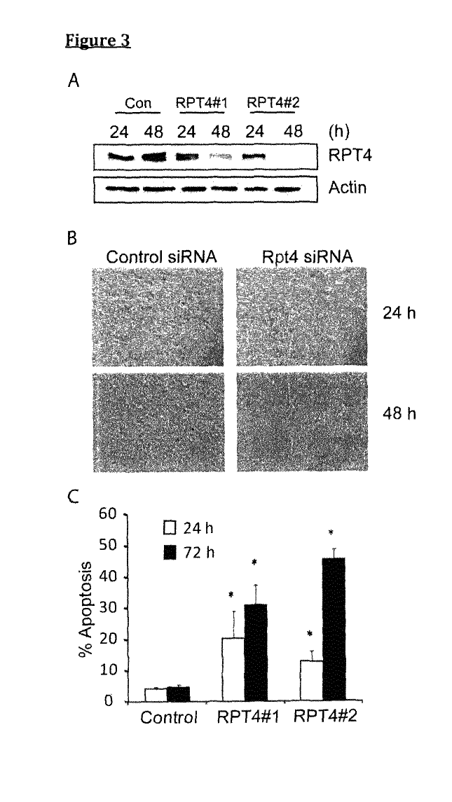

FIG. 3: (A) Effect of treatment with two different siRNAs targeting RPT4 on RPT4 protein levels in HCT116 colon cancer cells for 24 and 48 h respectively. Protein levels were examined using Western blotting. Control received a scrambled RNA sequence. (B) Representative phase contrast images of cells transfected with RPT4 siRNA sequence #2 or Control siRNA at 24 and 48 h. (C) Quantification of apoptosis following treatment with siRNAs targeting RPT4 at indicated time periods. The amount of apoptosis was determined by Annexin V and Propidium Iodide staining using flow cytometry. Data are mean+/−SEM from n=3 experiments per time condition. *p<0.05 compared to Control siRNA treated cells (ANOVA post-hoc Tukey).

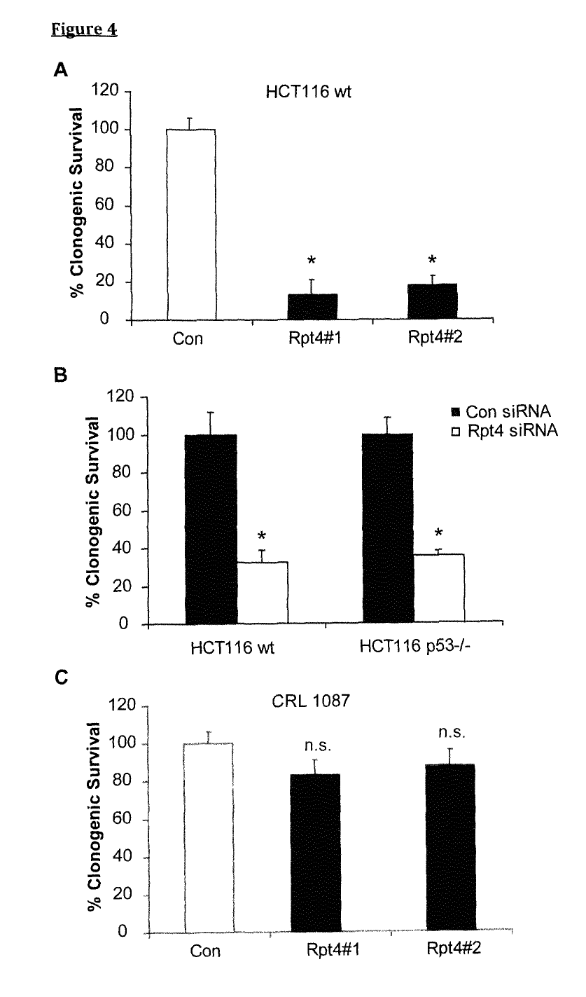

FIG. 4. siRNA-mediated reduction in RPT4 protein levels strongly impairs growth and survival of HCT 116 colon cancer cells whilst non-transformed colonocytes are unaffected. (A) HCT116 wild type (wt) cells transfected with two different siRNAs targeting RPT4 for 48 h and colongenic survival performed. Data are mean+/−SEM from n=3 experiments per time condition. *p<0.05 compared to Control siRNA treated cells (ANOVA post-hoc Tukey). (B) HCT116 wild type (wt) and p53 deficient (HCT116 p53−/−) cells transfected with a siRNA targeting RPT4 for 48 h and colongenic survival performed. Data are mean+/−SEM from n=3 experiments per time condition. *p<0.05 compared to Control siRNA treated cells (ANOVA post-hoc Tukey). (C) Non-transformed colonocytes, CRL-1807, were transfected with two different siRNAs targeting RPT4 for 48 h and colongenic survival performed. Data are mean+/−SEM from n=3 experiments per time condition. n.s.; not significant.

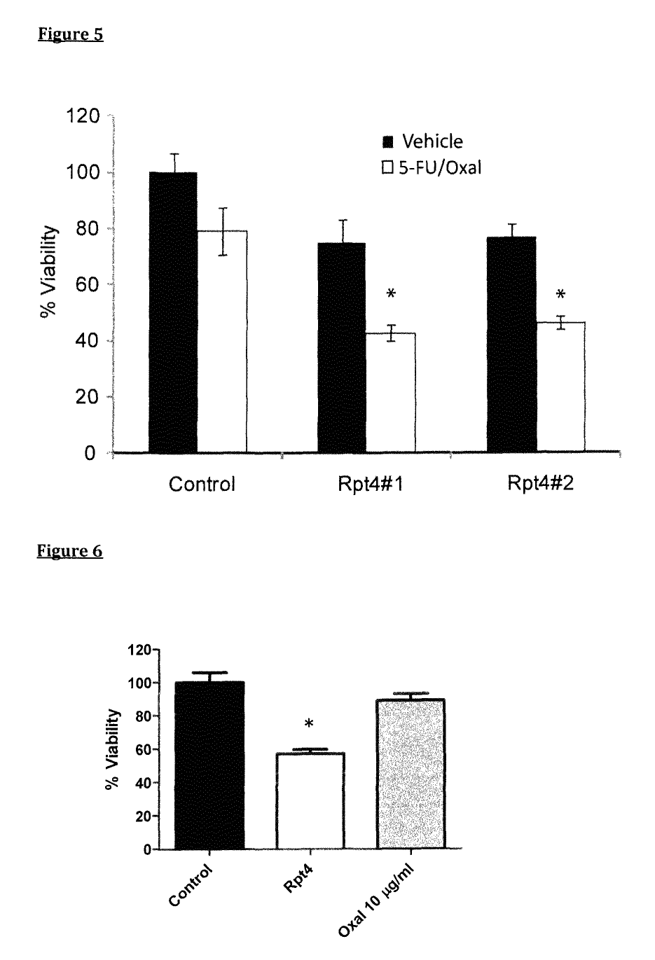

FIG. 5. RPT4 gene silencing enhances cytotoxicity to 5-fluorouracil and oxaliplatin in a colorectal cancer cell line. HCT 116 cells were treated with either control scrambled siRNA or one of two siRNA sequences to RPT4. After 48 hours, these cells were treated with either vehicle or a combination of 5-fluorouracil (30 μg/ml) and oxaliplatin (10 μg/ml). 48 hours later, a cell viability assay was carried out by measuring MTT reduction. Cellular proliferation was markedly reduced in cells treated with a combination of cytotoxic agents and RPT4 gene silencing than either treatment alone. Data represents mean+/−sem. (n=4); *p<0.05, ANOVA post-hoc Tukey.

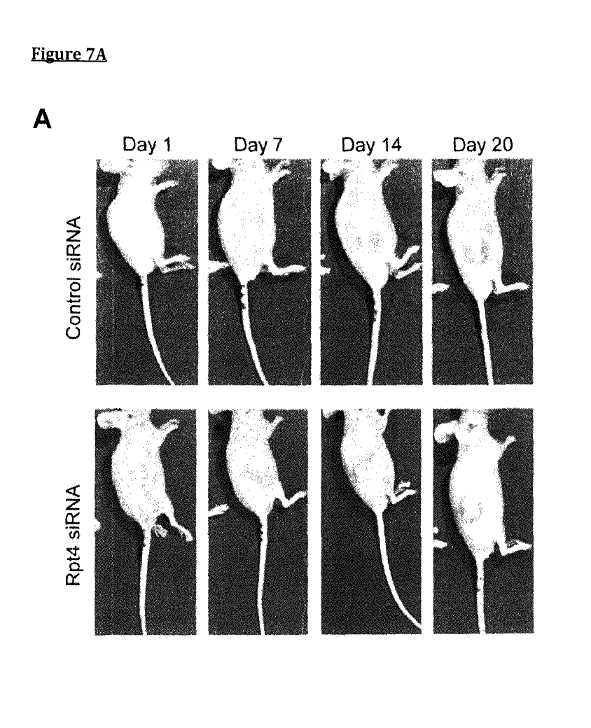

FIG. 6. Inhibition of RPT4 expression in an oxaliplatin resistant cell line reduces cell proliferation and survival. HCT116 cells were rendered oxaliplatin resistant by a stepwise addition of increasing concentration of oxaliplatin over a period of 16 passages. At the end of this the calculated IC50 for the HCT116 cells was 0.35 μM compared to 5 μM in the oxaliplatin resistance HCT116 cells. These oxaliplatin resistant HCT116 colon cancer cells were treated with either control siRNA (black column) or RPT4 siRNA (white column) for 48 h. Cell proliferation and viability was assessed by MTT assay. To confirm the resistance of the cell line to oxaliplatin, the cells were treated with 10 μg/ml of oxaliplatin for 48 h (grey column). Data are means+/−SEM from n=6 cultures per treatment. *p<0.05 compared to Control siRNA treated cultures (ANOVA post-hoc Tukey).

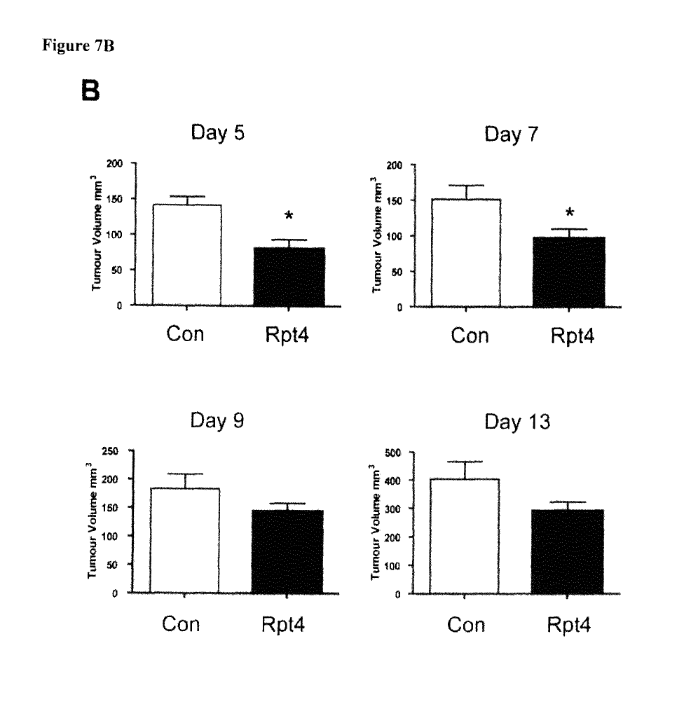

FIG. 7. Silencing of RPT4 expression modulates tumour growth in vivo. (A) Tumour growth in vivo. Bioware ULTRA HCT 116 WT-luc2 colon cancer cells were transfected ex vivo with either RPT4 siRNA (100 nM) or control scrambled siRNA (100 nM). Forty eight hours after transfection, these cells were suspended in BD Matrigel and D-PBS (1:1) and 2.5×10⁶ cells were implanted by subcutaneous injection to the right flank of Balb/c nude mice. Images were obtained weekly. Day 1 corresponds to 1 day post-inoculation. (B) Average tumour volume. Tumour dimensions were obtained at regular intervals with calipers, and tumour volume calculated using the modified ellipsoidal formula. Results represent average tumour volume in control group and group inoculated with RPT4-treated cells. Data reflect the mean+/−sem of n=10 mice (C) Kaplan Meier survival analysis of tumour bearing mice implanted with control scrambled siRNA or RPT4 siRNA treated colon cancer cells. Survival was prolonged in the group implanted with cells treated with RPT4 siRNA. (p=0.026, log-rank test).

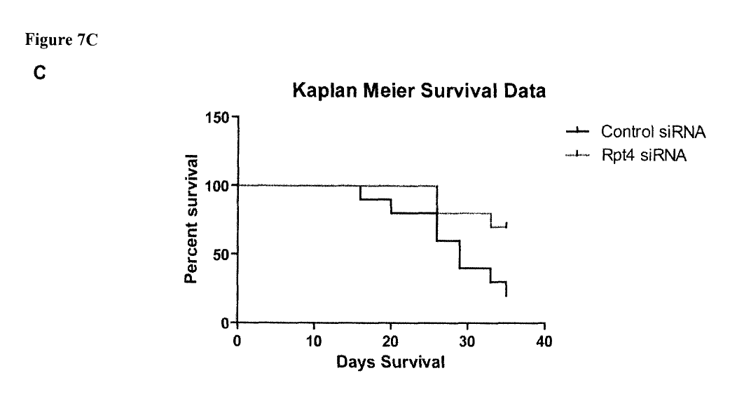

### DETAILED DESCRIPTION OF THE INVENTION

In a large-scale translational research project funded through Cancer Research Ireland and the Health Research Board (‘APOCOLON’—Study), the Applicants have identified a selective enrichment of the proteasomal subunit Rpt4 in the tumour tissue of CRC patients when compared to adjacent normal tissue. In contrast to RPT4, most other proteasomal subunits were equally expressed in normal and cancer tissues. The Applicants furthermore demonstrate herein that depletion of cellular RPT4 levels using low-molecular weight siRNAs dramatically reduced the survival and growth of colon cancer cells, but has negligible effects in non-transformed cells. Inhibition of RPT4 also reduced survival and growth of colon cancer cells that are defective in p53 signalling. Loss of p53 function occurs in the majority of CRC patients. The development of siRNAs targeting RPT4 or the development of small molecule RPT4 inhibitors there represent novel approaches for the treatment of CRC that specifically target a gene that is overexpressed in human colorectal cancer, and that is of vital importance for tumour growth and survival.

The invention is based on the surprising finding that RPT4, one of six RPT proteins located in the 19S subunit of the UPP, can function as a therapeutic target for solid tumours, and function as a prognostic variable of outcome in solid tumours. While the literature described a putative link between UPP activity and solid tumours, to date there is no published literature providing a link between RPT4 and treatment or prognostics of solid tumours.

Accordingly, the invention provides a method for treatment of a cancer, especially a solid tumour cancer, in particular a colorectal solid tumour, optionally a chemotherapeutic-resistant tumour, which comprises a step of decreasing the activity of RPT4 in tumour cells. The activity may be decreased in a number of different ways which will be apparent to a person skilled in the art, including reducing the expression of the protein (for example by means of low molecular weight inhibitors such siRNA or shRNA), or by directly inhibiting the activity of the protein by administering a RPT4 inhibitor or an antibody that has specific binding affinity for RPT4.

The term “RPT4” refers to a human protein that forms part of the 19S subunit of the UPP, and is one of a family of RPT proteins, RPT1-6.

In this specification, the term “treating” refers to administering a RPT4 inhibitor, optionally in combination with a conventional chemotherapeutic agent, to an individual that has a cancer, typically a solid tumour cancer, with the purpose to cure, heal, prevent, alleviate, relieve, alter, remedy, ameliorate, or improve the cancer or symptoms of the cancer. When the term is applied to the use of a RPT4 inhibitor and a conventional chemotherapeutic agent, the respective active agents may be administered together, or separately, and may be administered at the same time or at different times. In one embodiment, the patient may be treated to a course of one active agent, which is then followed by treatment with a course of the second active agent. The term “therapeutically effective amount” refers to the amount of the RPT4 inhibitor or chemotherapeutic agent/therapy that is required to confer the intended therapeutic effect in the individual, which amount will vary depending on the type of inhibitor, route of administration, status of cancer, and possible inclusion of other therapeutics or excipients.

The methods of the invention apply especially to solid tumour cancers (solid tumours), which are cancers of organs and tissue (as opposed to haematological malignancies), and ideally epithelial cancers. Examples of solid tumour cancers include pancreatic cancer, bladder cancer, prostate cancer, ovarian cancer, colorectal cancer (CRC), breast cancer, renal cancer, lung cancer, hepatocellular cancer, cervical cancer, gastric cancer, esophageal cancer, head and neck cancer, melanoma, neuroendocrine cancer. Suitably, the solid tumour cancer suitable for treatment and prognosis according to the methods of the invention are selected from CRC, breast and prostate cancer. In a preferred embodiment of the invention, the invention relates to treatment and prognosis of CRC. In another aspect, the methods of the invention apply to treatment and prognosis of outcome of haematological malignancies, including for example multiple myeloma, T-cell lymphoma, B-cell lymphoma, Hodgkins disease, non-Hodgkins lymphoma, acute myeloid leukemia, and chronic myelogenous leukemia.

The term “individual in need thereof” refers to a person who has or is suspected of having or developing established cancer, typically a person who has been diagnosed by a clinician as having Stage II, III or IV cancer.

To practice the methods of this invention, the above-described pharmaceutical composition can be administered orally, parenterally, by inhalation spray, topically, rectally, nasally, bucally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. A sterile injectable composition, e.g., a sterile injectable aqueous or oleaginous suspension, can be formulated according to techniques known in the art using suitable dispersing or wetting agents (such as Tween 80) and suspending agents. The sterile injectable preparation can also be a sterile injectable solution or suspension in a non-toxic parentally acceptable diluents or solvent for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (eg. Synthetic mono- or dyglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluents or dispersant, or carboxymethyl cellulose or similar dispersing agents. Other commonly used surfactants such as Tweens or Spans or other similar emulsifying agents or bioavailablity enhancers which are commonly used in the manufacture of pharmaceutically acceptable solid, liquid, or other dosage forms can also be used for the purposes of formulation.

A composition for oral administration can be any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavouring, or colouring agents can be added. A nasal aerosol or inhalation composition can be prepared according to techniques well known in the art of pharmaceutical formulation. A fused multicyclic compound-containing composition can also be administered in the form of suppositories for rectal administration.

The carrier in the pharmaceutical composition must be “acceptable” in the sense of being compatible with the active ingredient of the formulation (and preferable, capable of stabilising it) and not deleterious to the subject to be treated. For example, one or more solubilising agents, which form more soluble complexes with the fused multicyclic compounds, or more solubilising agents, can be utilised as pharmaceutical carriers for delivery of the active compounds. Examples of other carriers include colloidal silicon dioxide, magnesium stearate, sodium lauryl sulphate, and D&C Yellow #10.

The term “RPT4 inhibitor” refers to a compound that is capable of decreasing the activity of RPT4 in vivo. The activity may be decreased in a number of different ways which will be apparent to a person skilled in the art, including reducing the expression of the protein (for example by means of low molecular weight inhibitors such as for example siRNA or shRNA), or by directly inhibiting the activity of the protein by administering a RPT4 inhibitor or an antibody that has specific binding affinity for RPT4 or a RPT4 subunit. In a preferred embodiment of the invention, the invention relates to a low molecular weight inhibitor of RPT4 expression, the details of which will be well known to the person skilled in the field of molecular biology, and which include siRNA, shRNA, miRNA, antisense oligonucleotides, and ribozyme molecules. Small inhibitory RNA (siRNA) are small double stranded RNA molecules which induce the degradation of mRNAs. Micro RNA's (miRNAs) are single stranded (−22 nt) non-coding RNAs (ncRNAs) that regulate gene expression at the level of translation. Alternatively, small hairpin RNA (shRNA) molecules are short RNA molecules having a small hairpin loop in their tertiary structure that may be employed to silence genes. The design of miRNA or shRNA molecules capable of silencing RPT4 will be apparent to those skilled in the field of miRNA or shRNA molecule design. As an alternative, the level of tumour RPT4 expression can be modulated using antisense or ribozyme approaches to inhibit or prevent translation of RPT4 mRNA transcripts or triple helix approaches to inhibit transcription of the RPT4 gene. Antisense approaches involve the design of oligonucleotides (either DNA or RNA) that are complementary to RPT4 mRNA. The antisense oligonucleotides will bind to the complementary mRNA transcripts and prevent translation. Ribozyme molecules designed to catalytically cleave RPT4 mRNA transcripts can also be used to prevent translation and expression of RPT4. (See, e.g., PCT International Publication WO90/11364, published Oct. 4, 1990; Sarver et al., 1990, Science 247: 1222-1225).

In one embodiment of the invention, the RPT4 inhibitor is a RPT4 antagonist. One example of a RPT4 antagonist is an anti-RPT4 antibody (i.e. an antibody which specifically binds to human RPT4 protein). An example of such an antibody is sold by Abcam under the catalogue number ab22639. RPT4-specific antibodies may be produced using methods which are generally known in the art. In particular, purified RPT4 may be used to produce antibodies or to screen libraries of pharmaceutical agents to identify those which specifically bind RPT4. Antibodies to RPT4 may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use. Single chain antibodies (e.g., from camels or llamas) may be potent enzyme inhibitors and may have advantages in the design of peptide mimetics, and in the development of immuno-adsorbents and biosensors (Muyldermans, S. (2001) J. Biotechnol. 74:277-302). For the production of antibodies, various hosts including goats, rabbits, rats, mice, camels, dromedaries, llamas, humans, and others may be immunized by injection with RPT4 or with any fragment or oligopeptide thereof which has immunogenic properties (especially the fragment specified above). Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol.

It is preferred that the oligopeptides, peptides, or fragments used to induce antibodies to RPT4 have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of RPT4 amino acids may be fused with those of another protein, such as KLH, and antibodies to the chimeric molecule may be produced. Monoclonal antibodies to RPT4 may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; and_Cole, S. P. et al. (1984) Mol. Cell. Biol. 62: 109-120.)

In addition, techniques developed for the production of “chimeric antibodies”, such as the splicing of mouse antibody genes to human antibody genes to obtain a molecule with appropriate antigen specificity and biological activity, can be used (see, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-454.). Alternatively, techniques described for the production of single chain antibodies may be adapted, using methods known in the art, to produce RPT4-specific single chain antibodies. Antibodies with related specificity, but of distinct idiotypic composition, may be generated by chain shuffling from random combinatorial immunoglobulin libraries (see, e.g., Burton, D. R. (1991) Proc. Natl. Acad. Sci. USA 88:10134-10137.). Antibodies may also be produced by inducing in vivo production in the lymphocyte population or by screening immunoglobulin libraries or panels of highly specific binding reagents as disclosed in the literature (see, e.g., Orlandi, R. et al. (1989) Proc. Natl. Acad. Sci. USA 86:3833-3837; Winter, G. et al. (1991) Nature 349:293-299).

Antibody fragments which contain specific binding sites for RPT4 may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity (see, e.g., Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between RPT4 and its specific antibody. A two-site, monoclonal-based immunoassay utilizing monoclonal antibodies reactive to two non-interfering RPT4 epitopes is generally used, but a competitive binding assay may also be employed (Pound, supra). Various methods such as Scatchard analysis in conjunction with radioimmunoassay techniques may be used to assess the affinity of antibodies for RPT4. Affinity is expressed as an association constant, Kₐ, which is defined as the molar concentration of RPT4-antibody complex divided by the molar concentrations of free antigen and free antibody under equilibrium conditions. The Kₐ determined for a preparation of polyclonal antibodies, which are heterogeneous in their affinities for multiple RPT4 epitopes, represents the average affinity, or avidity, of the antibodies for RPT4. The Kₐ determined for a preparation of monoclonal antibodies, which are monospecific for a particular RPT4 epitope, represents a true measure of affinity. High-affinity antibody preparations with Kₐ ranging from about 10⁹ to 10¹² L/mole are preferred for use in immunoassays in which the RPT4-antibody complex must withstand rigorous manipulations.

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of RPT4-antibody complexes. Procedures for evaluating antibody specificity, titer, and avidity, and guidelines for antibody quality and usage in various applications, are generally available.

The invention provides a method of treating a cancer, especially a solid tumour cancer, or increasing the sensitivity (or reducing the resistance) of a cancer, typically a solid tumour, to a conventional chemotherapeutic agent, comprising a step of administering to the individual a therapeutically effective amount of a RPT4 inhibitor in conjunction with administration of a therapeutically effective amount of the conventional chemotherapeutic agent. The RPT4 inhibitor may be administered together with the chemotherapeutic agent (for example at the same time or as part of a single dose), or it may be administered in advance of or after administration of the chemotherapeutic agent. The term “conventional chemotherapeutic agent” as employed herein should be understood to mean a chemotherapeutic agent which is generally conventionally employed as a first line treatment for solid tumours, such as for example: antimetaboliotes including capecitibine, gemcitabine, 5-fluorouracil, fludarabine, cytarabine and mercaptopurine; topoisomerase inhibitors including doxorubicin; vinca alkaloids; taxanes including paclitaxel; platinum agents including cisplatin, oxaliplatin and carboplatin, irinotecan, leucovir, avastin, cetuximab. In this context, the term “therapeutically effective amount” typically refers to an amount of RPT4 inhibitor which increases the sensitivity (or decreases the resistance) of the tumour cell to the chemotherapeutic agent compared to a tumour cell which has not be treated with a RPT4 inhibitor.

The invention also relates to a method of treating chemotherapeutic-resistant cancer, especially a chemotherapeutic-resistant solid tumour cancer, in an individual in need thereof which involves administering to the individual a therapeutically effective amount of a RPT4 inhibitor, for example a low molecular weight inhibitor of RPT4, along with a therapeutically effective amount of a conventional chemotherapeutic agent, for example 5-FU/oxaliplatin or 5-FU/irinotecan. The term “chemotherapeutic-resistant solid tumour” is an art-recognised term and should be understood to mean a tumour whose volume does not decrease following treatment with the chemotherapeutic agent/therapy. This is common is advanced colorectal cancer solid tumours.

The invention also relates to the use of tumour expression levels of RPT4 as a prognostic variable of outcome in a solid tumour cancer. The term “outcome” means the likelihood that the individual will die as a result of the cancer, and/or when it is likely to occur. In particular, the term “outcome” refers to the likelihood of disease-free survival. The prognostic variable is the abundance of RPT4 protein expression in tumour cells, and can be determined by measuring RPT4 expression levels (i.e. at a RNA level) or by direct determination of protein abundance (for example by means of an ELISA test). Thus, increased abundance of RPT4 correlates with reduced disease free survival and death in node positive individuals.

### Materials and Methods:

### Cancer Tissue Specimens for Western Blot Analysis

All tissue was sourced from the tissue bio-bank at Beaumont Hospital, Dublin 9, Ireland. A cohort of patients with Stage III colon cancer was selected from the Bowel Cancer database. Informed consent was obtained from patients. Colonic resections were sent fresh to the Pathology Department where a trained technician obtained tumour and adjacent normal tissue specimens which were subsequently snap frozen. Pathology reports for the colon tumours were then made available. Ethical approval to work with cancer specimens was granted at the Beaumont Hospital Ethics Committee meeting.

### Tissue Microarray and Immunohistochemical Analysis of RPT4 as a Prognostic Marker

Tissue microarrays (TMAs) were constructed from 228 colorectal cancer cases taken from a phase III trial of adjuvant 5-fluorouracil—based chemotherapy compared with postoperative observation alone. The study group consisted of 228 non-consecutive patients with demographic variables including gender and age at surgery recorded. Histological stage II and III, tumour grade, TMN stage and presence or absence of vascular invasion were included in the analysis and whether the patients were randomised to adjuvant chemotherapy with 5-FU or no treatment was noted. Time to disease progression, including age at recurrence, site of recurrence and time to recurrence were also verified. Sections 4 μm in thickness were cut from array blocks and floated onto adhesive slides. Sections were then baked at 55° C. overnight. All staining was carried out on a BondMax automated immunostainer from Vision BioSystems. Sections were loaded onto the system and the relevant program was started. The BondMax system dewaxed the slides and then carried out and antigen retrieval was performed. Sections were stained with an antibody directed against RPT4 (Enzo Life Sciences). Immunostained slides were scored for RPT4 expression using a standardised scoring system. Each slide was evaluated using light microscopy with respect to intensity. An intensity score was assigned that represents the average intensity of the positive cells (0=no staining; 1=weak; 2=moderate; 3=strong). Slides were scored by two independent pathologists blinded to the clinic-pathological data and entered into a database. At the time of analysis, a median follow of 6.5 years available for analysis. Univariate and multivariate analysis was carried out using logistic regression and Cox's proportional hazard model. Univariate analysis of overall and progression-free survival was performed by the Kaplan-Meier method and log-rank test and carried out in SPSS for Windows 15.0. A p-value of less than 0.05 was considered significant.

### Cell Culture

HCT116 wild type and HCT116 p53−/− cells were cultured in RPMI 1640 containing 10% fetal bovine serum, 2 mM L-glutamine, 100 U/ml penicillin, and 100 mg/ml streptomycin, in a humidified atmosphere of 5% CO₂ in air at 37° C. Non-transformed colonocytes, CRL-1807, were obtained from the American Type Culture Collection (ATCC) and were maintained in DMEM containing 10% fetal bovine serum, 100 U/ml penicillin, and 100 mg/ml streptomycin, in a humidified atmosphere of 5% CO₂ in air at 37° C.

### Western Blot Analysis

1 ml of lysis buffer [50 mmol/L HEPES (pH 7.5), 150 mmol/L NaCl, 5 mmol/L Na-EDTA] was added to each tissue sample. The samples were lysed on ice and homogenised using the Ultra-Turrax T25 Basic Homogeniser. 30 second pulses were used to break down the tissue. Protein concentrations were determined using the standard Pierce Micro-BCA Protein Assay (Pierce, Northumberland, UK). Standard SDS-polyacrylamide gel electrophoresis (SDS-PAGE) was performed. Protein samples were prepared for electrophoresis by denaturing at 95° C. for 10 min in the presence of 2×SDS loading buffer (100 mM Tris-Cl, pH 6.8, 4% SDS, 0.2% bromophenol blue, 20% glycerol). 20 μg of protein was loaded onto SDS polyacrylamide gels which were suspended in a chamber of running buffer (25 mM Tris-Cl, pH 8.3, 250 mM glycine and 0.1% SDS) Protein marker was also loaded which allowed identification of proteins of different sizes. Proteins were transferred onto a 0.2 μM nitrocellulose membrane (Schleicher and Schuell, Germany) using the Bio-Rad's Trans-Blot SD Semi-Dry Electrophoresis Transfer Cell following electrophoresis. and 0.01 6% SDS). The nitrocellulose membrane was initially blocked for 60 min in blocking solution (5% non-fat milk in 0.1% TBS-T (1 mM Tris-Cl, pH 8.0, 15 mM NaCl, 0.005% Tween) and then washed three times in 0.1% TBST. The primary antibody was prepared at the required concentration in the milk blocking solution (1:1000). The secondary antibody was diluted 1:10000. Proteins were detected by briefly exposing the membrane to equal volumes of Amersham ECL chemiluminescent detection reagent (RPN2105, Amersham Biosciences, UK). The membrane was then transferred to an exposure cassette, and images obtained by using the FujiFilm Image Reader Las-3000.

### Clonogenic Survival Assay

To examine the capability of parental versus RPT4 knockdown cells to survive and proliferate, a clonogenic survival assay was performed (Franken et al., 2006). HCT-116 cells were seeded at 7×104 in RPMI media in a 24 well plate for 24 hours. At 24 hours, control siRNA, and 2 varients of siRNA against RPT4 were added to 8 wells respectively. At 24 and 48 hours, the cells were trypsinised and counted using the Neubauer haemocytometer. 1000 cells were then reseeded into a new 60 mm tissue culture dish in triplicate and incubated for 9 days in regular media. At day 9, the media was removed and 1 ml of Clonogenic Reagent (50% Ethanol, 0.25% 1,9-dimethyl-methylene blue) was added. The plates were left at room temperature for 45 minutes. Following incubation with the clonogenic reagent, plates were washed twice with PBS. Colony formation was quantified after staining with methylene blue (Sigma-Aldrich). Digital Images were subsequently acquired of the plates.

siRNA Knockdown of RPT4

We performed gene-specific targeting of RPT4 corresponding to nucleotides 81-99 (GGAGUUAAGGGAACAAUUA)—SEQUENCE ID NO: 1 and 174-192 (UGAAGUGCUUAAACAGUUA)—SEQUENCE ID NO: 2 of the coding sequence. As a negative control cells were treated with a non-targeting sequence that does not lead to specific degradation of any known cellular genes. Sequences were then obtained from Sigma Proligo.HCT-116 cells were seeded 4×10⁵ and allowed to recover over night. Cells were transfected with 50 nm of the siRNA in the presence of metafectene in serum-free media for 4 hours. RPMI media was added following 4 hours. Following transfection, cells were harvested at 24 hr and 48 hr. Cells from each sample were recovered in preparation for Immunoblotting and subsequent analysis.

### Flow Cytometry

Apoptosis can be measured using this method which allows high throughput analysis in real time. We examined the amount of externalised phosphatidylserine (PS) and DNA using the binding of Annexin V and Propidium Iodide (PI) as indicators. Annexin V allows identification of cell surface changes that occur in early apoptosis. Cells were then collected with trypsin-EDTA. Next, cells were centrifuged at 1000 rpm×3 min and supernatant was removed leaving a cell pellet. Subsequently cells were washed twice in PBS and resuspended in 100 μl of binding buffer (10 mM Hepes, 140 mM NaCl, 2.5 mM Ca Cl2) containing Annexin-V FITC conjugated (5 μl/ml) (BioVision, Mountain View, Calif., USA) and Propidium Iodide (PI) (1 μg/ml) and for 20 min at room temperature prior to flow cytometric analysis. Cells were then resuspended in ice-cold binding buffer. Flow cytometry was performed on a Partec Cyflow ML16 flow cytometer (Partec, Munster, Germany) equipped with a 488 nm argon ion laser, 532 nm diode laser and a 405 nm diode laser.

### Screen for Rpt4 Inhibitors

Recombinant Rpt4 will expressed and purified as a soluble GST-tagged fusion protein in E. coli for utilisation in a luciferase based high-throughput assay for monitoring Rpt4 activity. Luciferase bioluminescence will be measured as a readout of ATP levels remaining following addition of varying concentrations of recombinant Rpt4. The amount of ATP remaining at the end of the assay will be inversely proportional to the activity of Rpt4. In this manner combinatorial libraries of small molecule inhibitors will be screened for Rpt4 inhibitory activity with luminescence signal serving as the readout for the assay.

### In-Vivo Studies

(A) The ability of Rpt4 siRNA to enhance the sensitivity of chemotherapy treatment regimes is assessed in an in vivo xenograft model of colon cancer in mice. In summary, 5 groups of mice (n=10 per group) undergo treatment as follows:
1. HCT116 cells implanted and treated in vivo with 5-fluorouracil and oxaliplatin.
2. HCT116 cells implanted and treated in vivo with nanoparticles/microparticles containing control siRNA
3. HCT116 cells implanted and treated in vivo with nanoparticles/microparticles containing Rpt4 siRNA.
4. HCT116 cells implanted and treated in vivo with nanoparticles/microparticles containing control siRNA and 5-fluorouracil and oxaliplatin.
5. HCT116 cells implanted and treated in vivo with nanoparticles/microparticles containing Rpt4 siRNA and 5-fluorouracil and oxaliplatin.

(B) In order to investigate the potential of Rpt4 as a monotherapy for chemoresistant tumours, the ability of Rpt4 inhibition to inhibit the growth and survival of chemoresistant cells xenografted into mice, and treated with the Rpt4 siRNA every 3 days for a period of one month, is tested. HCT116 cells rendered resistant to 5-fluorouracil and oxaliplatin by standard protocols are implanted into mice and tumour growth is assessed and life span analysis is performed to investigate whether the Rpt4 inhibition can modulate tumour survival in chemoresistant cells compared to a control siRNA sequence.

The invention is not limited to the embodiments hereinbefore described which may be varied in construction and detail without departing from the spirit of the invention.

1. A method for treating a solid tumour, the method comprising: administering a low molecular weight inhibitor of RPT4 in combination with one or more conventional solid tumour chemotherapeutic agents.

2. The method of claim 1, wherein the solid tumor is a colorectal solid tumour.

3. The method of claim 1, wherein the low molecule weight inhibitor of RPT4 comprises an siRNA inhibitor of RPT4.

4. The method of claim 1, wherein the one or more conventional solid tumour chemotherapeutic agents comprises an antimetabolite, a topoisomerase inhibitor, a vinca alkaloid, a taxane, a platinum agent, a therapeutic antibody, or a combination thereof.

5. The method of claim 4, wherein the one or more conventional solid tumour chemotherapeutic agents comprises 5-FU and a second conventional solid tumor chemotherapeutic agent selected from the group consisting of an antimetabolite, a topoisomerase inhibitor, a vinca alkaloid, a taxane, a platinum agent, and a therapeutic antibody.

6. The method of claim 5, wherein the second conventional solid tumor chemotherapeutic agent is oxaliplatin or irinotecan.

7. The method of claim 3, wherein the one or more conventional solid tumour chemotherapeutic agents comprise 5-FU and a second conventional solid tumour chemotherapeutic agent selected from the group consisting of oxaliplatin and irinotecan.

8. A pharmaceutical composition comprising a therapeutically effective amount of a low molecular weight RPT4 inhibitor, and one or more conventional solid tumour chemotherapeutic agents, in combination with a pharmaceutically acceptable excipient.

9. The pharmaceutical composition of claim 8, wherein the low molecular weight RPT4 inhibitor is a low molecular weight inhibitor of RPT4 expression selected from the group consisting of an siRNA, an shRNA, an miRNA, an antisense, and a ribozyme.

10. The pharmaceutical composition of claim 9, wherein the one or more conventional solid tumour chemotherapeutic agents are selected from the group consisting of: an antimetabolite, a topoisomerase inhibitor, a vinca alkaloid, a taxane, a platinum agent, and a therapeutic antibody.

11. The pharmaceutical composition of claim 10, wherein the one or more conventional solid tumour chemotherapeutic agents comprises 5-FU in combination with a second conventional solid tumour chemotherapeutic agent selected from the group consisting of: an antimetabolite, a topoisomerase inhibitor, a vinca alkaloid, a taxane, a platinum agent, and a therapeutic antibody.

12. The pharmaceutical composition of claim 11, wherein the composition comprises 5-FU/oxaliplatin or 5-FU/irinotecan, in combination with an RPT4 siRNA molecule.

13. A method for treating a chemotherapeutic resistant solid tumor, the method comprising administering a low molecular weight inhibitor of RPT4 to a subject having a chemotherapeutic resistant solid tumor.

14. The method of claim 13, wherein the low molecular weight inhibitor of RPT4 is a siRNA molecule.

15. The method of claim 13, wherein the solid tumour is a colorectal solid tumour.

16.-17. (canceled)

18. A method for increasing the sensitivity of a solid tumour cell to a conventional chemotherapeutic agent, the method comprising administering to a subject a composition comprising an RPT4 inhibitor in conjunction with one or more conventional chemotherapeutic agents.

19. The method of claim 18, wherein the RPT4 inhibitor is a low molecular weight inhibitor of RPT4 expression selected from the group consisting of: an siRNA, an shRNA, an miRNA, an antisense, and a ribozyme.

20. The method of claim 18, wherein the one or more conventional chemotherapeutic agents are selected from the group consisting of an antimetabolite, a topoisomerase inhibitor, a vinca alkaloid, a taxane, a platinum agent, and a therapeutic antibody.

21. The method of claim 18, wherein the one or more conventional solid tumour chemotherapeutic agents comprise 5-FU in combination with an antimetabolite, a topoisomerase inhibitor, a vinca alkaloid, a taxane, a platinum agent, or a therapeutic antibody.

22. The method of claim 21, wherein the composition comprise 5-FU/oxaliplatin or 5-FU/irinotecan, in combination with an RPT4 siRNA.

23. A method of estimating outcome for a solid tumour cancer patient, the method comprising a step of determining a level of expression of RPT4 in a tumour cell from the patient, in which increased expression of tumour RPT4 in the tumour cell compared with a non-tumour cell correlates with reduced disease free survival and/or death.

24.-30. (canceled)


SEQUENCE LISTING (2 sequences)

SEQ ID NO: 1 - RNA, 19 nt, Homo sapiens
ggaguuaagggaacaauua

SEQ ID NO: 2 - RNA, 19 nt, Homo sapiens
ugaagugcuuaaacaguua